(19) United States
(12) Patent Application Publication
(10) Pub. No.: US 2005/0008609 A1
(43) Pub. Date: Jan. 13, 2005

(54) Multi-component reverse thermo-sensitive polymeric systems

(75) Inventors: Daniel Cohn, Jerusalem (IL); Alejandro Sosnik, Jerusalem (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(73) Assignees: YISSUM RESEARCH DEVELOPMENT COMPANY OF THE HEBREW UNIVERSITY OF JERUSALEM Hi Tech Park, The Hebrew; University Of Jerusalem, Jerusalem (IL)

(21) Appl. No.: 10/789,431

(22) Filed: Feb. 27, 2004

Related U.S. Application Data
(63) Continuation of application No. PCT/IL02/00699, filed on Aug. 22, 2002.
(60) Provisional application No. 60/314,640, filed on Aug. 27, 2001.

(30) Foreign Application Priority Data
Aug. 15, 2002 (IL) 151288

(51) Int. Cl. A61K 31/74

(52) U.S. Cl. 424/78.1

(57) ABSTRACT

A multi-component environmentally responsive polymeric system, having at least two environmentally responsive polymeric components capable of undergoing a transition that results in a sharp increase in viscosity in response to a change in temperature at a predetermined body site, wherein the at least two components display different reverse thermal gelation behavior in the human body.

### RELATED APPLICATIONS

This application is a continuation of International Application No. PCT/IL02/00699 filed Aug. 22, 2002, the contents of which are here incorporated by reference in their entirety, and claims priority under 35 USC 120 therefrom, and claims priority from provisional U.S. Application No. 60/314,640, filed Aug. 27, 2001, incorporated herein by reference in its entirety, and from Israeli Application No. 151288 filed Aug. 15, 2002.

### BACKGROUND OF THE INVENTION

1. Field of the Invention

The present invention discloses a new type of multi-component polymeric systems displaying superior reverse thermal gelation (RTG) behavior, comprising more then one reverse thermo-sensitive polymer, for the purposes of performing in various areas, preferably in the biomedical field.

2. Prior Art

There is a wide variety of materials which are foreign to the human body and which are used in direct contact with its organs, tissues and fluids. These materials are called Biomaterials, and they include, among others, polymers, ceramics, biological materials, metals, composite materials and combinations thereof.

The development of polymers suitable to be implanted without requiring a surgical procedure, usually named injectable polymers, has triggered much attention in the last years. These materials combine low viscosity at the injection stage, with a gel or solid consistency developed in situ, later on. The systems of the present invention are preferably used, without limitation, as matrices for the controlled release of biologically active agents, as sealants, as coatings and as barriers in the body. The area of Tissue Engineering represents an additional important field of application of the improved responsive systems disclosed hereby, where they can perform as the matrix for cell growth and tissue scaffolding.

The syringability of injectable biomedical systems is their most essential advantage, since it allows their introduction into the body using minimally invasive techniques. Furthermore, their low viscosity and substantial flowability at the insertion time, allow them to reach and fill spaces, otherwise unaccessible, as well as to achieve enhanced attachment and improved conformability to the tissues at the implantation site. On the other hand, the sharp increase in viscosity is a fundamental requirement for these materials to be able to fulfill any physical or mechanical function, such as sealing or performing as a barrier between tissue planes. The high viscosities attained play also a critical role in generating syringable materials that, once at the implantation site, are also able to control the rate of release of drugs or can function as the matrix for cell growth and tissue scaffolding.

Clearly, biodegradability is yet another important requirement for some of these materials.

A polymer network is characterized by the positive molecular interactions existing between the different components of the system. These interactions may be physical in nature, such as chain entanglements, or chemical such as ionic interactions, hydrogen bonding, Van der Waals attractions and covalent bonding. Bromberg et al. (U.S. Pat. No. 5,939,485) developed responsive polymer networks exhibiting the property of reversible gelation triggered by a change in diverse environmental stimuli, such as temperature, pH and ionic strength. The gels are useful in a variety of medical applications including drug delivery.

The term “thermosensitive” refers to the capability of a polymeric system to achieve significant chemical, mechanical or physical changes due to small temperature differentials. The resulting change is based on different mechanisms such as ionization and entropy gain due to water molecules release, among others (Alexandridis and Hatton, Colloids and Surfaces A, 96, 1 (1995)). Since one of their fundamental advantages is to avoid the need for an open surgical procedure, thermo-responsive materials are required to be easily syringable, combining low viscosity at the injection stage, with a gel or solid consistency being developed later on, in situ.

Thermosensitive gels can be classified into two categories: (a) if they have an upper critical solution temperature (UCST), they are named positive-sensitive hydrogels and they contract upon cooling below the UCST, or (b) if they have a lower critical solution temperature (LCST), the are called negative-sensitive hydrogels and they contract upon heating above this temperature.

The reverse thermo-responsive phenomenon is usually known as Reversed Thermal Gelation (RTG) and it constitutes one of the most promising strategies for the development of injectable systems. The water solutions of these materials display low viscosity at ambient temperature, and exhibit a sharp viscosity increase as temperature rises within a very narrow temperature interval, producing a semi-solid gel once they reach body temperature. There are several RTG displaying polymers. Among them, poly(N-isopropyl acrylamide) (PNIPAAm) (Tanaka and co-workers in U.S. Pat. No. 5,403,893 and Hoffman A. S. et al., J. Controlled Release, 6, 297 (1987)).

Unfortunately, poly(N-isopropyl acrylamide) is non-degradable and, in consequence, is not suitable for a diversity of applications where biodegradability is required. Additionally, the N-isopropylacrylamide monomer is toxic.

Undoubtedly, one of the most relevant examples of RTG-displaying polymers is the family of poly(ethylene oxide)/poly(propylene oxide)/poly(ethylene oxide) (PEO-PPO-PEO) triblocks, commercially available as Pluronic™ (Krezanoski in U.S. Pat. No. 4,188,373). Adjusting the concentration of the polymer, renders the solution with the desired liquid-gel transition. Nevertheless, relatively high concentrations of the triblock are required (typically above 15-20%) to produce compositions that exhibit such a transition, even minor, at commercially or physiologically useful temperatures. An additional system which is liquid at room temperature, and becomes a semi-solid gel when warmed to about body temperature, is described in U.S. Pat. No. 5,252,318, and consists of tetrafunctional block polymers of polyoxyethylene and polyoxypropylene condensed with ethylenediamine (commercially available as Tetronic.™)

The endothermic phase transition taking place, is driven by the entropy gain caused by the release of water molecules bound to the hydrophobic groups in the polymer backbone. Unfortunately, despite of their potential, some fundamental performance drawbacks severely restrict their clinical use. Therefore, even though these materials exhibit a significant increase in viscosity when heated up to 37° C., the levels of viscosity attained are not high enough for most clinical applications. Derived from this fundamental limitation, these systems do not display satisfactory mechanical properties and the residence times achieved at the implantation body site are unacceptably short. Furthermore, due to these characteristics, these gels have high permeabilities, a property which renders them unsuitable for drug delivery applications because of the fast drug release kinetics of these gels. Despite of their clinical potential, these materials have failed to be used successfully in the clinic, because of serious performance limitations (Steinleitner et al., Obstetrics and Gynecology, 77, 48 (1991) and Esposito etal., Int. J. Pharm. 142, 9 (1996)).

Biodegradability is the process whereby the molecular weight of polymers decreases because of repeated chain scission, due to hydrolytic and/or enzymatic attack until, ultimately, dissolution takes place. This phenomenon plays a fundamental role in a diversity of devices, implants and prostheses, since it avoids the need to remove the system, once it has accomplished its objectives. In addition, they can perform as matrices for the release of bioactive molecules and result in improved healing and tissue regeneration processes. Biodegradable polymers such as polyesters of a-hydroxy acids, like lactic acid or glycolic acid, are used in diverse applications such as bioabsorbable surgical sutures and staples, some orthopedic and dental devices, drug delivery systems and more advanced applications such as the absorbable component of selectively biodegradable vascular grafts, or as the temporary scaffold for tissue engineering. Biodegradable polyanhydrides and polyorthoesters having labile backbone linkages, have been developed, the disclosures of which are incorporated herein. Polymers which degrade into naturally occurring materials, such as polyaminoacids, also have been synthesized. Degradable polymers formed by copolymerization of lactide, glycolide, and s-caprolactone have been disclosed. Polyester-ethers have been produced by copolymerizing lactide, glycolide or c-caprolactone with polyethers, such as polyethylene glycol (“PEG”), to increase the hydrophilicity and degradation rate.

Unfortunately, the few absorbable polymers clinically available today are stiff, hydrophobic solids which are, therefore, clearly unsuitable for non-invasive surgical procedures, where injectability is a fundamental requirement. The only way to avoid the surgical procedure with these polymers, is to inject them as micro or nanoparticles or capsules, typically containing a drug to be released. As an example, injectable implants comprising calcium phosphate particles in aqueous viscous polymeric gels, were first proposed by Wallace et al. in U.S. Pat. No. 5,204,382. Even though these the ceramic component is generally considered to be nontoxic, the use of non-absorbable particulate material seems to trigger a foreign body response both at the site of implantation as well as at remote sites, due to the migration of the particles, over time.

Among the approaches developed, the in situ precipitation technique developed by R. Dunn, as disclosed in U.S. Pat. No. 4,938,763, is one strategy worth mentioning. These systems comprise a water soluble organic solvent, in which the polymer is soluble. Once the system is injected, the organic solvent gradually diffuses into the aqueous biological medium, leaving behind an increasingly concentrated polymer solution, until the polymer precipitates, generating the solid implant in situ. A similar approach has been reported by Kost et al (J. Biomed. Mater. Res., 50, 388-396 (2000)).

In situ polymerization and/or crosslinking is another important technique used to generate injectable polymeric systems. Hubbell et al described in U.S. Pat. No. 5,410,016, water soluble low molecular precursors having at least two polymerizable groups, that are syringed into the site and then polymerized and/or crosslinked in situ chemically or preferably by exposing the system to UV or visible radiation. Mikos et al (Biomaterials, 21, 2405-2412 (2000)) described similar systems, whereas Langer et al (Biomaterials, 21, 259-265 (2000)) developed injectable polymeric systems based on the percutaneous polymerization of precursors, using UV radiation. An additional approach was disclosed by Scopelianos and co-workers in U.S. Pat. No. 5,824,333 based on the injection of hydrophobic bioabsorbable liquid copolymers, suitable for use in soft tissue repair.

All these techniques have serious drawbacks and limitations, which significantly limit their applicability. The paradox in this area has to do, therefore, with the large gap existing between the steadily increasing clinical demand for Injectables, on one hand, and the paucity of materials suitable to address that need, on the other hand.

### SUMMARY OF THE INVENTION

Each of the different components of the invention disclosed hereby may be in a variety of forms, including, without limitation, in their respective water solution form. The present invention covers also compositions where all the materials or part of them are initially in their solid form (particles, fibers, fabrics, foam-like structures, etc.) dissolving in due time, and as they dissolve, they gradually contribute to the RTG performance of the system. The contribution of the gradually dissolving constituent may affect the properties of the system in various ways, including, without limitation, resulting in an increase or decrease in its viscosity, affect its life span, as well as fundamentally influence its biological performance.

The structures and devices disclosed hereby, capitalize on combining, in a unique and advantageous way, two or more reverse thermo-sensitive polymers, to obtain novel and superior water based RTG systems of broad applicability, preferably in the biomedical field.

The term ‘thermosensitive’ refers to the capability of a polymeric system to achieve significant chemical, mechanical or physical changes due to small temperature differentials. The compositions disclosed hereby are tailored-made and capitalize on the uniqueness of the Reverse Thermal Gelation phenomenon. The endothermic phase transition taking place, is mainly driven by the entropy gained because of the release of water molecules bound to the hydrophobic groups in the polymer backbone. Its clear, therefore, that the balance between the hydrophilic and hydrophobic moieties in the molecule, as well as molecular weight considerations and chain mobility parameters, play a crucial role. Consequently, the properties of the different compositions disclosed by the present invention, are adjusted and balanced by variations of the basic chemistry, molecular weight and physical state of the different components.

The unique and essential feature of the present invention is the presence of more than one polymeric reverse thermo-responsive component capable of undergoing a transition that results in a sharp increase in viscosity in response to a change in temperature at a predetermined body site, wherein said at least two components display different reverse thermal gelation behavior.

The two components may comprise different reverse thermo-responsive polymers in any of their possible forms, e.g., solutions of different concentrations, solids of different geometries, etc., or the same polymer but at different concentrations or in a different state, i.e., a water solution as opposed to a solid. This, in fundamental contrast to the RTG systems of the prior art, in which only one component produces the solution exhibiting the viscosity increase, as temperature raises. Clearly, the distinction between this invention and the prior art is not merely quantitative, but one of essence, since the presence of more than one RTG-displaying polymer in the compositions disclosed hereby, renders these systems with significantly different properties than those of the prior art and allows to attain performance characteristics unattainable with the prior art.

One of the unique features of the RTG compositions disclosed hereby is their ability to display tailored, time-dependent viscosity profiles, an RTG behavior unattainable by the systems of the prior art. Each of the components of the system displays a distinct RTG behavior, with its own Ti (the temperature at which the system gels and the viscosity increases sharply) and specific characteristics, including, without limitation, their rheological and transport properties, as dictated by the requirements of any given application. This applies to both the constituents that are already in their water solution form at time t=O, as well as those constituents that are in their solid form at time t=O, being gradually solubilized in situ, with time.

The solid component or components appear in a diversity of shapes, sizes and geometries, including, without limitation, spheres, particles of any other shape, capsules, fibers, ribbons, films, meshes, fabrics, non-woven structures, foams, porous structures of different types, each of them having the possibility of being solid, porous, hollow and/or combinations thereof. The initially solid component or components may differ significantly in their behavior and in their different properties, including, without limitation, their composition as well as their physical, rheological and mechanical characteristics.

When more than one RTG-displaying polymer is initially present in its solid form, the system may be engineered in various different configurations and combinations thereof. Among others, and without limitation, the compositions disclosed hereby may consist of different particles, each type comprising a different RTG-displaying polymer and the particles are then mixed together. Also, each particle, regardless of its shape, size and geometry and other parameters, may combine more than one component in a simple blended manner or may be engineered so that a diversity of spatial arrays, are generated. These include, without limitation, layered structures, core-sheath structures and domains-continuous matrix structures, as well as other types of spatial arrangements, such as radial or circumferential arrays, among others.

The diverse components of the invention disclosed hereby are preferably different reverse thermo-sensitive polymers, as described above, but they may also consist of co-polymeric systems of various types, comprising segments displaying a distinct RTG behavior, with its own Ti and specific Theological properties. This applies to both the constituents that are already in their water solution form at insertion time, as well as those constituents that are solid at the beginning of their use, being solubilized in situ, with time.

The materials and the water solutions disclosed hereby, are advantageously used in a diversity of clinical areas, including, without limitation, their use as injectables in non-invasive or minimally invasive surgery, in the area of Tissue Engineering, in the prevention of post-surgical adhesions, in the field of Gene Therapy and as matrices for the controlled release of biologically active molecules.

The process whereby the multi-component compositions are produced, is yet another variable of the present invention. For example, and without limitation, the incorporation of the different constituents into the system can be done following various schemes, such as being added simultaneously or sequentially, below or above their respective temperatures of gelation (Ti), each of the components being added in one or various shots or dropwise, or each of the components being added under different conditions, or alternately, or aiming at generating diverse spatial arrays, among many others.

The system can be of various types, differing in several of their characteristics, including, without limitation, the basic polymeric RTG materials used, as well as the number and form of each of the components present. Also, they may differ in the size and shape of each of the RTG phases, the characteristics of the interphase generated between them, and their rheological properties, among other aspects. Also, the invention hereby disclosed comprises non-biodegradable materials, biodegradable ones or combinations thereof. The initially solid component or components may be crosslinked or not.

The multi-constituent compositions of the present invention include combinations of any type of reverse thermo-responsive materials selected from a group consisting of commercially available poly(ethylene oxide)-poly(propylene oxide)-poly(ethylene oxide) (PEO-PPO-PEO) triblocks, random or alternating reverse thermo-responsive PEO-PPO block copolymers as described e.g:, by Cohn and Sosnik, in Israeli Patent Specification No. 148,886 (the teachings of which are incorporated herein by reference), N-alkyl substituted acrylamides (preferably poly-N-isopropyl acrylamide [PNIPAAm]), cellulose derivatives, selected from a group consisting of hydroxypropyl methylcellulose and hydroxypropyl cellulose, alternating or random.

Also, the compositions of the present invention can be generated by combining different families of materials including, for example and without limitation, a system consisting of polyNIPAAm and PEO-PPO-PEO triblocks, among many others.

The compositions of the present invention may include, in addition to two or more reverse thermo-sensitive components, in their diverse forms, also polymers that are responsive to other stimuli, such as pH changes, ionic strength, electric and magnetic fields, fluids and biological species. Furthermore, in addition to the components described above, the compositions of the present invention may include also other materials that fulfill other roles, including, without limitation, rendering the system with the desired mechanical behavior or with the appropriate transport properties or with any other chemical, physical or biological characteristics, and combinations thereof. The compositions of the present invention may include, in addition to the diverse components described above, also materials that may contribute to the time-dependent viscosity profile of the composition, even though they do not display reverse-thermoresponsive behavior.

The multi-constituent nature of the compositions disclosed hereby, is intrinsic and unique to the invention, and plays a fundamental role in the development of novel systems for a broad range of areas. For example, and without limitation, in the case of systems delivering biologically active molecules, including drugs, among many others, a multi-modal release profile can be tailored into the system, where the initially solid RTG-polymer/s perform/s as a drug reservoir, releasing the drug very slowly, while the drug incorporated into water solution containing the RTG-polymer/s delivers the drug/s at a faster rate.

In addition to the tailoring versatility derived directly from the variety of compositions disclosed by the present invention, as well as to the geometrical variations available, the rate of release may be slowed down even further, by various ways, and combinations thereof. For example and without limitation, the rate of release can be retarded by crosslinking the initially solid RTG-polymer/s, with various types of crosslinkers, preferably with a biodegradable crosslinker, and by controlling its composition, structure, molecular weight and concentration in the polymer. Also, regardless of its size, shape and geometry, the solid material/s can be coated with numerous coating materials, preferably biodegradable, such as poly(lactic acid) or poly(caprolactone) among many others, to generate a transient barrier for the release of the biologically active molecule or molecules. The kinetics of the release of the biologically active molecule/s can be fine tuned also by crosslinking the surface layer of the particle both chemically as well as by exposing it to radiation of various types, such as gamma radiation or performing various types of surface plasma treatments, among others.

Also, as it dissolves, the initially solid RTG-polymer/s may change important properties of the solution, including, without limitation, its pH or its ionic strength or some biological parameter. For example, in one such scenario, it may increase the Ti of a component or various components present in the system, lowering, therefore, its or their viscosity at 37 degrees centigrade.

The multi-constituent nature of the compositions disclosed hereby, is intrinsic and unique to the invention, and plays a fundamental role in the development of novel systems for a broad range of areas, including, without limitation, the field of Tissue Engineering.

The objective of Tissue Engineering is to induce regeneration of functional tissue, by providing the appropriate three-dimensional scaffolding construct on which cells will be able to grow, differentiate and generate new tissue. Clearly, the composition and mechanical properties of the materials, strongly affect the ability of the system to actively promote the regeneration of autologous functional tissue. In addition, the macrostructural characteristics of the scaffold, play also a fundamental role in determining the type of cells and other tissular components present in the new tissue. Also, for a scaffold to perform successfully, it is required to be biocompatible, to display the right porosity and to be mechanically suitable. All of the above, aiming at achieving the essential goal of the template, namely, to perform as an adhesive substrate for cells, promoting their growth and differentiation, while retaining cell function, and inducing the regeneration of autologous functional tissue.

The template's ultimate task is to provide a gradually disappearing, temporary construct for the generation of viable new tissue. Therefore, if autologous tissue is to regenerate and replace the scaffold, until the invention disclosed hereby, biodegradability was one of its indispensable attributes.

Until now, the need for a transient scaffold resulted, necessarily, in constructs based on Biodegradable Polymers, such as poly(glycolic acid), poly(lactic acid) and copolymers of the two. Since the composition and architecture of natural tissues are greatly affected by the stress field induced by the scaffold, a clear dependency exists between the mechanical behavior of the construct and that of the regenerated tissue. In contrast to requirements, available Biodegradable Polymers are hydrophobic and rather stiff materials, generating rigid structures. Furthermore, their degradation results inevitably in two additional detrimental phenomena: a significant drop in the local pH and the generation of particulate material, triggering phagocytosis, irritating the tissue and interfering with the healing process. Consequently, the nature of these polymers both chemically as well as mechanically represent substantial disadvantages used as scaffolds for Tissue Engineering. It is apparent, therefore, that a new generation of templates for the regeneration of tissue, is called for.

The multicomponent systems of the present invention can be used advantageously as both the scaffold as well as the matrix.

The unique advantage of RTG-displaying matrices for Tissue Engineering derives directly from the viscosity differential inherent to their reverse thermo-responsive nature and allows its incorporation into the scaffolding structure as a very low viscosity water solution. Its only when the temperature of the system is raised above its Tᵢ, that the viscosity will increase sharply and gel. This will allow the incorporation of the cells into the system in a gentle and controlled way. The various parameters of the gel, most importantly Tᵢ and the viscosity of the gel at 37° C., can be easily fine tuned.

The uniqueness of scaffolds consisting of RTG-displaying polymers, not crosslinked or comprising biodegradable crosslinks, pertains not only to their mechanical properties and enhanced hydrophilicity but also to the way the construct will disappear. As opposed to the biodegradable polymers being currently used, these scaffolds will he able to gradually revert both the crosslinking and gelling processes. As a result, the scaffold can be “programmed” to liquefy over time, fading away following a pathway devoid of the important drawbacks germane of normal biodegradation processes. The various characteristics of the scaffold, including its water content, hydrated mechanical properties and the timing of the different stages, can be controlled. The “fading out” of the scaffold can be programmed into the system or triggered externally by gradually lowering the temperature a few degrees or by progressively shifting the Ti of the material so it becomes higher than body temperature.

Aiming at illustrating the applicability of the invention in this additional important area, and without limiting in any manner or fashion the scope of the invention, cells of different types can be incorporated into the various constituents of the compositions disclosed hereby, performing as water-rich matrices for cell growth and tissue regeneration. Each of the RTG-displaying water phases may contain one or more different types of cells aiming at affecting the biological performance of the systems, in different ways and/or at different points in time. The cells may also affect the environment of their own aqueous phase as well that of other cells, by cell metabolism or cells secretions. Cells may affect various properties of the medium, such as its pH, ionic strength and mineral balance, among others, and/or affect the activity of other components of the system, including enzymes, cells and genes, among others.

The scaffold itself is based on RTG materials selected from a group consisting of a diversity of shapes, sizes and geometries. The scaffolding structures consisting of reverse-thermoresponsive polymers may include, without limitation, spheres, particles of any other shape, capsules, fibers, ribbons, films, meshes, fabrics, non-woven structures, foams, porous structures of different types, each of them having the possibility of being solid, porous, hollow and/or combinations thereof. Different components of the scaffold may differ significantly in their behavior and in their different properties, including, without limitation, their composition as well as their physical, rheological and mechanical characteristics. The RTG-exhibiting scaffolding structure has the same design and performance versatility of all the initially-solid RTG-displaying components of the present invention, as described hereinabove.

Aiming at illustrating one multi-component Tissue Engineering system, the unique compositions of the present invention may comprise one or more components that are present, from the outset, in their water solution form, and/or initially solid RTG-displaying polymer/s and/or a scaffolding structure consisting of one or more RTG-displaying polymers, and combinations thereof. Also, the system may comprise yet an additional solid component in various forms, such as microparticles, among many others, that will dissolve at a specific point in time. The timely dissolution of this solid component would affect the properties of the medium and by that, trigger diverse processes. Examples of these processes can be, among numerous others, the fast release of a biologically active molecule, or the change of the pH of the solution, affecting, therefore, its viscosity, or speed up the dissolution of the scaffolding structure, and combinations thereof.

For the sake of clarity and simplicity, and without limiting the scope of the invention in any form or fashion, the inventors have chosen to illustrate the invention disclosed hereby, by focusing on a specific biomedical application and exemplifying the invention using two particular families of RTG polymers. This, even though the multi-component systems of the present invention include all families of RTG-displaying materials, and the polymers and solutions disclosed hereby, can be applied to numerous sites in the body and used in fundamentally different applications.

The application selected for illustrating this invention, is their use as injectables in non-invasive or minimally invasive surgical procedures.

Without limiting the scope of the invention in any form or fashion, two groups of polymeric reverse thermo-responsive compositions have been chosen by the inventors to illustrate the present invention: [1] the first group is based on the commercially available Pluronic.™ poly(ethylene oxide)-poly(propylene oxide)-poly(ethylene oxide) (PEO-PPO-PEO) triblocks and more specifically Pluronic F127 and [2] polymeric materials of the following generic formula [-Xₙ-A-Xₙ-E-B-E-]ₘ, wherein segments A are bifunctional, trifunctional or multifunctional hydrophilic segments, segments B are bifunctional, trifunctional or multifunctional hydrophobic, segments X are bifunctional degradable segments; wherein E are bi, tri or multifunctional chain extenders or coupling molecules, and wherein n and m denote the respective degrees of polymerization and y designates the additional functionality of the segment above 2.

More specifically in said polymeric materials:
A) A is a hydrophilic bifunctional segment selected from a group consisting of —OH, —SH, —COOH, —NH₂, —CN or —NCO group terminated poly(oxoethylene) or any other bifunctional hydrophilic segment having the appropriate terminal group, or a trifunctional segment selected from a group consisting in poly(oxoethylene triol), poly(oxoethylene triamine), poly(oxoethylene triacarboxylic acid), ethoxylated trimethylolpropane, or any other trifunctional hydrophilic segment having the appropriate terminal group, or other multifunctional segment, most preferably bifunctional, and/or combinations thereof;
B) B is a hydrophobic bifunctional component is selected from a group consisting of a —OH, —SH, —COOH, —NH₂, —CN or —NCO group terminated polyoxyalkylene polymer (selected from a group consisting of poly(propylene glycol) (PPG), polyoxopropylene diamine (Jeffamine.™), polytetramethylene glycol (PTMG)), polyesters selected from a group consisting of poly(caprolactone), poly(lactic acid), poly(glycolic acid) or combinations or copolymers thereof, polyamides or polyanhydrides or any other bifunctional hydrophobic segment having the appropriate terminal group, or a trifunctional segment selected from a group consisting of poly(oxopropylene triol), poly(oxopropylene triamine), poly(oxopropylene triacarboxylic acid), or any other trifunctional hydrophobic segment, having the appropriate terminal group, or other multifunctional hydrophobic segment, most preferably bifunctional segment, and combinations thereof;
C) E is a chain extender or coupling molecule is bifunctional reactive molecule selected from a group consisting of phosgene, aliphatic or aromatic dicarboxylic acids or any other reactive derivative (selected from a group consisting of oxalyl chloride, malonyl chloride, succinyl chloride, glutaryl chloride, fumaryl chloride, adipoyl chloride, suberoyl chloride, pimeloyl chloride, sebacoyl chloride, terephtaloyl chloride, isophtaloyl chloride, phtaloyl chloride and/or mixtures thereof or other dicarboxylic acid derivative), aminoacids selected from a group consisting of glycine, alanine, valine, phenylalanine, leucine, isoleucine or any other essencial aminoacid or not, oligopeptides selected from a group consisting of RGD, RGD-S or any other oligopeptide having or not biological activity, aliphatic or aromatic diamines selected from a group consisting of ethylene diamine, propylene diamine, butylene diamine, or any other diamine or amine

derivative, aliphatic or aromatic diols selected from a group consisting of ethylene diol, propanediol, butylenediol or any other diol, aliphatic or aromatic diisocyanates selected from a group consisting of hexamethylene diisocyanate, methylene bisphenyldiisocyanate, methylene biscyclohexanediisocyanate, tolylene diisocyanate or isophorone diisocyanate or any other bifunctional reactive molecule, having the appropriate terminal group or trifunctional reactive molecules selected from a group consisting of cyanuric chloride, triisocyanates, triamines, triols, aminoacids selected from a group consisting of lysine, serine, threonine, methionine, asparagine, glutamate, glutamine, histidine or any other essencial aminoacid or not having three functional groups, oligopeptides or any other trifunctional reactive molecule, having the appropriate terminal groups or multifunctional couplig molecule, most preferably phosgene, diisocyantes, aminoacids, oligopeptides or bifunctional carboxylic acid derivatives, and combinations thereof. E may also comprise combinations of the functional groups described above in the same molecule. The reaction products are poly(ether-carbonate)s, poly(ether-ester)s, poly(ether-urethane)s or derivatives of

chlorotriazine, most preferably poly(ether-carbonate)s, poly(ether-ester)s or poly(ether-urethanes), polyimides, polyureas and combinations thereof; and
A) Segment X renders the molecule degradable due to its hydrolytic instability and is based preferably on segments selected from a group consisting of aliphatic or aromatic ester, amide or anhydride groups formed from a-hydroxy carboxylic acid units or their respective lactones, selected from a group consisting of lactide, glycolide or 6-caprolactone, their respective lactams or the respective poly(anhydride)s.

The X segments comprise preferably hydroxy carboxylic units or their respective lactones, or similar compounds selected from a group and without limitation consisting of lactic acid, lactide, ε-caprolactone, glycolic acid, glycolide, β-propiolactone, δ-glutarolactone, δ-valerolactone, β-butyrolactone, ethylene carbonate, trimethylene carbonate, γ-pivalactone, α,α-diethylpropiolactone, p-dioxanone, 1,4-dioxepan-2-one, 3-methyl-1,4dioxanone-2,5-dione, 3,3-dimethyl-1,4-dioxanone-2,5-dione, cyclic esters of α-hydroxybutyric acid, α-hydroxyvaleric acid, α-hydroxyisovaleric acid, α-hydroxycaproic acid, α-hydroxy-α-ethylbutyric acid, α-hydroxyisocaproic acid,

α-hydroxy-α-methylvaleric acid, α-hydroxypentanoic acid, α-hydroxystearic acid, α-hydroxylignoceric acid, salycilic acid and mixtures, thererof or amino carboxylic units, such as caprolactam, laurolactam, lactamide and mixtures, thereof.

Aqueous solutions of the polymers of this invention display from slight to remarkable reverse thermal gelation characteristics: they combine the properties of low viscosity liquids at low temperatures (preferably around RT), with intermediate to high viscosities body temperature.

Focusing on a specific biomedical application and exemplifying the invention using two particular families of RTG polymers is intended only to illustrate preferred embodiments and should not be construed as limiting in any way or fashion, the scope of this invention, as more broadly set forth hereby.

The areas of applicability of the compositions of the present invention include, without limitation, their use as matrices for the controlled release of biologically active agents, as sealants, as coatings and lubricants and as transient barriers in the body aiming at reducing or preventing of adhesions subsequent to surgical procedures. The area of Tissue Engineering represents an additional important field of application of these materials, where they can perform as both the matrix and the scaffold for cell growth and tissue regeneration. The compositions disclosed hereby can be used in the Tissue Engineering field in both schemes, when the whole process takes place in vivo, as well as when it is initially conducted in vitro followed by the implantation of the system.

It is an object of the invention to engineer structures and devices which combine, in a unique and advantageous way, two or more reverse thermo-sensitive components, to obtain novel and superior water based RTG systems of broad applicability, preferably in the biomedical field.

It is an object of this invention to generate RTG-displaying systems able to display tailored, time-dependent viscosity profiles, this behavior being unattainable by the systems of the prior art.

It is also an object of this invention to incorporate the diverse components of the invention in a variety of forms, including, without limitation, in their water solution form.

It is also an object of this invention to generate RTG-displaying systems in which all its components or part of them are initially in their solid form, dissolving in due time, and as they dissolve, they gradually contribute to the RTG performance of the system.

It is also an object of this invention to generate RTG-displaying systems where the solid component or components appear in a diversity of shapes, sizes and geometries, including, without limitation, spheres, particles of any other shape, capsules, fibers, ribbons, films, meshes, fabrics, non-woven structures, foams, porous structures of different types, each of them having the possibility of being solid, porous, hollow, and/or combinations thereof.

It is also an object of this invention to generate RTG-displaying systems where the initially solid component or components may differ significantly in their behavior and in their different properties, including, without limitation, their composition as well as their physical, rheological and mechanical characteristics.

It is an additional object of the invention to generate multi-component RTG-displaying polymeric systems, comprising non-biodegradable materials, biodegradable ones or combinations thereof. In the case of biodegradable systems, these materials are engineered to display different degradation kinetics.

It is also an object of the invention to introduce hydrolytically unstable segments along the polymeric backbone, allowing, therefore, to fine tune both the degradation rate of the polymer molecule as well as control various properties of the system, including their stability and rheological properties.

It is also an object of the invention to introduce hydrolytically unstable segments in the crosslinking bonds, allowing, therefore, to revert to a non-crosslinked system regaining, thereby, its reverse thermo-responsive properties, minimized or lost because of having been crosslinked. This allows, in a given scenario, the injection of the syringable system, that will gel at 37° C. and then crosslink in situ, attaining superior properties. In due time, the crosslinks will degrade, reverting to the uncrosslinked state, where a drop in tempertature will allow the gel to liquify.

It is an additional object of the invention to generate multi-component RTG-displaying polymeric systems, where the various reverse thermo-sensitive polymers initially present in their solid form, can have different configurations and combinations thereof. Among others, and without limitation, the compositions disclosed hereby may consist of different particles, each type comprising a different RTG-displaying polymer and the particles are then mixed together. Also, each particle, regardless of its shape, size and geometry and other parameters, may combine more than one component in a simple blended manner or may be engineered so that a diversity of spatial arrays, are generated. These include, without limitation, layered structures, core-sheath structures and domains-continuous matrix structures.

It is an additional object of the invention to generate multi-component RTG-displaying polymeric systems, that may include, in addition to two or more reverse thermo-sensitive polymers, in their diverse forms, also polymers that are responsive to other stimuli, such as pH changes, ionic strength, electric and magnetic fields, fluids and biological species. It is an additional object of the present invention to generate multi-component RTG-displaying polymeric systems, that may include, in addition to two or more reverse thermo-sensitive polymers, also other materials that fulfill other roles, including, without limitation, rendering the system with the desired mechanical behavior or with the appropriate transport properties or with any other chemical, physical or biological characteristics, and combinations thereof. It is an additional object of the present invention to generate multi-component RTG-displaying polymeric systems, that may include, in addition to the diverse components described above, also materials that may contribute to the time-dependent viscosity profile of the composition, even though they do not display reverse-thermoresponsive behavior.

It is also an object of the invention to generate multi-component RTG-displaying polymeric systems, that can be used advantageously in a diversity of clinical areas, including, without limitation, their use as injectables in non-invasive or minimally invasive surgery, as sealants, in the areas of Tissue Engineering and Gene Therapy, in the prevention of post-surgical adhesions, and as matrices for the controlled release of molecules of biological relevance.

It is an additional object of the invention to generate multi-component RTG-displaying polymeric systems, that can be used advantageously in the area of Tissue Engineering, performing as both the matrix as well as the scaffold.

It is an additional object of the invention to render these compositions with specific biological functions by incorporating biomolecules of various types, including, without limitation, drugs, enzymes, proteins, peptides, growth factors, and hormones, by blending them into the system or by binding them covalently or otherwise to the polymer. The compositions disclosed hereby can comprise more than one type of drug, for different therapeutic purposes, or for the same therapeutic objective, but at different points in time. Furthermore, since different phenomena appear at different times, different drugs may be incorporated within RIG-displaying polymeric components that differ in diverse characteristics, including, without limitation, their composition, the viscosity of the solution generated, their physical state (for example, still solid as opposed to already in solution), and, for the case of solid components, their size and shape. The versatility of the compositions disclosed hereby, allow to tailor the drug or drugs release profile or profiles in a rather independent and versatile way.

It is an additional object of the invention to incorporate cells of various types into these materials, for them to perform as RTG-displaying matrices for cell and tissue growth. It is an additional object of the invention to incorporate cells of various types into these materials, for them to perform as RIG-displaying scaffolding materials in the field of Tissue Engineering and other areas, for the purpose of cell growth and tissue regeneration.

Thus according to the present invention, there is provided a multi-component environmentally responsive polymeric system, comprising at least two environmentally responsive polymeric components capable of undergoing a transition that results in a sharp increase in viscosity in response to a change in temperature at a predetermined body site, wherein said at least two components display different reverse thermal gelation behavior in the human body.

The term “different reverse thermal gelation behavior” as used herein is intended to denote inter alia that the different components attain different viscosities at 37° C., that they have different Ti values, meaning that their viscosities raise at different temperatures, and that one may have to dissolve over time before it starts to be RTG relevant.

In addition, in preferred embodiments of the present invention, said at least two

components display different reverse thermal gelation behavior, displaying initially a defined interface between them, i.e., the components have different RTG properties as a function of two parameters, namely time and position in space within the sample. Clearly, as time elapses the interface will progressively disappear. Thus, in embodiments having a liquid-liquid boundary, it will fade away with time, as the two liquid phases “equilibrate” and diffuse one into the other, while in embodiments having a solid-liquid interface, it will gradually disappear as the solid dissolves into the liquid phase. In both cases of these types of preferred embodiments, initially there will be a defined boundary/interface between the two or more RTG components of the system.

In preferred embodiments of the present invention, each of said components is comprised of the same polymer and said components are present in different concentrations and as a result of said different concentrations display different reverse thermal gelation behavior. In said preferred embodiments of the present invention, each of said components is comprised of the same polymer and said components preferably present in different states, already dissolved in water and as a solid, and as a result of said different states display different reverse thermal gelation behavior.

In further preferred embodiments of the present invention, said responsive polymeric system comprises at least two different environmentally responsive polymeric components.

Shih and Zentner (WO 00 66085) (D3) claim in claim 1 “A dual phase polymeric agent-delivery composition comprising: (a) a continuous biocompatible gel phase, (b) a discontinuous particulate phase comprising defined microparticles, and (c) an agent to be delivered contained in at least said discontinuous particulate phase.” In claim 9, the inventors claim that: “ . . . said continuous gel phase is formed from a reverse thermal gelation (RTG) system comprising an effective amount of block copolymers comprising biodegradable hydrophobic polyester A polymer blocks and polyethylene glycol B polymer blocks.” and in claim 10 they claim “The composition according to claim 9 wherein said RTG system is a mixture of two or more said block copolymers having different gelation properties.” Aiming at clarifying their invention, the inventors clearly state in the Detailed Description of the Invention section, in page 6, row 29 and below, the following: “RTG mixture or “blend of RTG” refers to an RTG system comprising a

blend of two or more types of triblock copolymers that have different block polymer ratios, molecular weights, gelation temperatures and the like.” The inventors move on and further clarify that: “The RTG mixture or blend can be made either by simply mixing two or more individual previously synthesized poly(ethylene glycol)s with lactide, glycolide, caprolactone, and the like, to form a new RTG system, or by reacting two or more block copolymers to synthesize the mixed system.” As apparent to anybody skilled in the art, these blended solutions are inherently homogeneous systems and will necessarily generate one homogeneous RTG solution, having one Ti value and one averaged out viscosity level. In striking contrast to the invention disclosed in WO 00 66085 (D3), the unique and essential feature of the present invention is the presence of more than one polymeric reverse thereto-responsive component capable of undergoing a transition that results in a sharp increase in viscosity in response to a change in temperature at a predetermined body site, wherein said at least two components display different reverse thermal gelation behavior. The term “different reverse thermal gelation behavior” as used herein is intended to denote inter alia that the different components attain different viscosities in the human body at 37° C., that they have different Ti values, meaning that their viscosities raise at different temperatures, and that one may have to dissolve over time before it starts to be RTG relevant. As also stated below, said at least two components display different reverse thermal gelation behavior, exhibiting a defined interface between them in the human body, i.e. the components have different RTG properties as a function of two parameters, namely time and position in space within the implanted sample. It is apparent, therefore that the invention disclosed in WO 00 66085 (D3) is in fundamental contrast to the invention disclosed herein.

In WO 97 10849 A (D4), in their amended claim 1, the inventors claim: “A polymeric micelle drug composition capable of solubilizing a hydrophobic drug, which comprises: a micelle of a block copolymer having an hydrophilic component and a hydrophobic component, and a hydrophobic drug physically incorporated into the micelle; wherein the hydrophobic component is a biodegradable polymer selected from the group consisting of polylactide, polyglycolide, poly(lactide glycolide), polycaprolactone, and a mixture thereof; and the hydrophilic component is poly(alkylene oxide).” Even though some of the polymers mentioned in claim 1 may, under specific conditions, display RTG properties, this invention does not relate in any form or manner to a reverse thermal gelation system and its only objective is to use amphiphilic block copolymers to generate micelles and solubilize diverse hydrophobic drugs within the hydrophobic rnicellar core. Furthermore, the systems disclosed in WO 97 10849 A (D4) are, as is all the prior art relevant to the invention disclosed herein, inherently homogeneous mixtures or blends of polymers co-dissolved in an aqueous medium that, should they display RTG behavior, would necessarily generate one homogeneous RTG solution, having one Ti value and one averaged out viscosity level. In striking contrast to the invention disclosed in WO 97 10849 A (D4), the unique and essential feature of the present invention is the presence of more than one polymeric reverse thermo-responsive component capable of undergoing a transition that results in a sharp increase in viscosity in response to a change in temperature at a predetermined body site, wherein said at least two components display different reverse thermal gelation behavior. The term “different reverse thermal gelation behavior” as used herein is intended to denote inter alia that the different components attain different viscosities in the human body at 37° C., that they have different Ti values, meaning that their viscosities raise at different temperatures, and that one may have to dissolve over time before it starts to be RTG relevant. As also stated below, said at least two components display different reverse thermal gelation behavior, exhibiting a defined interface between them in the human body, i.e. the components have different RTG properties as a function of two parameters, namely time and position in space within the implanted sample it is apparent, therefore that the invention disclosed in WO 97 10849 A (D4), is in fundamental contrast to the invention disclosed herein.

Martini et al, Journal of the Chemical society, Fraraday Transaction, Royal Society of Chemistry, vol. 90, no, 13, Jul. 7, 1994, pp. 1961-1966 (D5) describe triblock copolymers comprising a central PEG segment and two lateral e-caprolactone short blocks. In their study, the authors describe blocks comprising a PEG4000 chain and caprolactone segments consisting of 2, 4 or 6 repeating units and investigated some of their properties, as a function of the concentration and temperature. All three

copolymers were studied separately and no attempt whatsoever was made to study mixtures of more than one polymer. In striking contrast to the data reported by Martini et al (D5), the unique and essential feature of the present invention is the presence of more than one polymeric reverse thermo-responsive component capable of undergoing a transition that results in a sharp increase in viscosity in response to a change in temperature at a predetermined body site, wherein said at least two components display different reverse thermal gelation behavior, The term “different reverse thermal gelation behavior” as used herein is intended to denote interalia that the different components attain different viscosities in the human body at 37° C., that they have different Ti values, meaning that their viscosities raise at different temperatures, and that one may have to dissolve over time before it starts to be RIG relevant. As also stated below, said at least two components display different reverse thermal gelation behavior, exhibiting a defined interface between them in the human body, i.e. the components have different RTG properties as a function of two parameters, namely time and position in space within the implanted sample. It is apparent, therefore that the data reported by Martini et al (D5), does not teach or suggest the invention disclosed herein.

Pilo and Chung (WO 01 82970 A1) (D1) state in the Background of the Invention section that: It would be desirable to provide a biodegradable reverse gelation system having a gelation temperature within a desired range so that the system remains as a liquid at an ambient temperature, but becomes a gel at the body temperature of the object to which the drug is delivered.” The authors indicate that such polymeric systems could be then “easily processed, formulated and dispensed at ambient temperatures, thereby significantly reducing manufacturing and handling costs. In addition, accidental gelation during application, e.g. gelation in the syringe during injection can be avoided. As discussed above, the gelation temperature of a reverse thermal gelation system may be modified by changing the chain length, the glycolide/lactide (GIL) mole ratio of the A-polymer block, the molecular weight of the B-polymer block, the weight ratio of A block and B block polymers, and by various additives. However, the above modifications also change the gel qualification as well as the gelation temperature. In addition, some additives may not be compatible with the drug to be delivered. Therefore, it is desirable to provide a reverse gelation system with adjustable gelation temperatures without changing its desirable gel qualities significantly. It has been discovered that mixtures or blends of two or more tri-block polyester/polyethylene glycol (PEG) copolymers provides for improved reverse thermal gelation properties, such as an optimum gelation temperature, gel strength, degradation rate, and yet stir maintains the desirable gel qualities.” claim I of WO 01 82970 A1 (D1) claims: “A biodegradable polymeric system possessing reverse thermal gelation properties comprising a mixture of at least a Component 1 triblock copolymer and a Component 11 triblock copolymer.” As defined by the authors in the Detailed Description of Preferred Embodiments of the Invention, “a mixture of triblock copolymers refers to a reverse gelation system comprising two or more ABA or more BAB triblock polyester-polyehtyelene glycol copolymer components. The mixture can be made either by simply mixing two or more individually synthesized triblock copolymer components, or by synthesizing two or more types of copolymer systems in one synthesizing vessel.” The inventors also state, in the first paragraph of this section that The individual triblock copolymer components can be synthesized separately and then mixed, or be synthesized by polymerization of two or more polyethyelene glycol polymers having different molecular weights in one reaction vessel.” Furthermore, the inventors state that “Polymer solution” or aqueous solution” and the like, when used in reference to a biodegradable block copolymer contained in that solution, shall mean a water based solution having such block copolymer mixtures or blends dissolved therein at a functional concentration, and maintained at a temperature below the gelation temperature of the block copolymer mixtures.’ As obvious to the person skilled in the art, such systems will inevitably, generate one homogeneous RTG solution having one gelation temperature (Ti) and one averaged out viscosity level. It is apparent, therefore, that the objective of the invention disclosed in WO 01 82970 AI (DI) is to be able fine tune the gelation temperature of the solutions generated by their PEG/PLA containing triblocks without affecting the characteristics of the gel considerably, and they do so by co-dissolving more than one triblock or by copolymerizing PEG chains of different length. Such solutions are inherently homogeneous and necessarily will create homogeneous RTG-displaying systems with average properties. This can be further illustrated by Examples 1 through 4, where in Examples 1 and 2 systems having Ti values of 13° C. and 42° C., respectively, are produced, while in Example 3 various mixtures of the previous polymers are prepared, covering the 13° C.-42° C. temperature interval. Furthermore, in Example 4 the 11 of the system is adjusted by simultaneously synthesizing two different triblock copolymer components in one reaction vessel”. In this example, PEG1450 and PEG1000 were used and “The gelling temperature of the polymer solution was 22° C.” In striking contrast to the invention disclosed in WO 01 82970 AI (D1), the unique and essential feature of the present invention is the presence of more than one polymeric reverse thermo-responsive component capable of undergoing a transition that results in a sharp increase in viscosity in response to a change in temperature at a predetermined body site, wherein said at least two components display different reverse thermal gelation behavior. The term “dfferent reverse thermal gelation behavior” as used herein is intended to denote inter alia that the different components attain different viscosities in the human body at 37° C., that they have dfferent Ti values, meaning that their viscosities raise at different temperatures, and that one may have to dissolve over time before it starts to be RTG relevant, As also stated below, said at least two components display different reverse thermal gelation behavior, exhibiting a defined interface between them in the human body, i.e. the components have different RTG properties as a function of two parameters, namely time and position in space within the implanted sample. It is apparent, therefore that the invention disclosed in WO 01 82970 AI (D1) is in fundamental contrast to the invention disclosed herein.

Shih and Zentner (WO 01 76558) (D2) claim in Claim I ‘A drug delivery system for controlled protein release into a biological environment comprising: (a) a sparingly soluble biocompatible particle; (b) an effective amount of a protein or peptide deposited onto the particle forming a substantially insoluble protein/particle combination; and (c) a biocornpatible polymeric matrix having dispersed therein the protein/particle combination,” in the Background of the invention section, the inventors indicate: In the prior art, attempts have been made to stabilize and/or reduce the solubility of proteins and peptides by complexing the proteins or peptides with multivalent cations such as zinc, calcium, magnesium, copper, ferric ion, and nickel, to name a few.” They also ndicate when related to the prior art that: “However, neither of these patents disclose the deposit of proteins or peptides onto biocompatible sparingly soluble particles in order to stabilize and/or prolong the release of proteins from a drug delivery biopolymer. Thus, it would be desirable to provide such a composition so that the solubility of the protein and/or the dissolution rate of protein from a drug delivery biopolymer device are reduced.” When relating to the gel, in claim 14 the inventors state that: “ . . . said biocompatible polymeric matrix is a block copolymer selected from the group consisting of ABA block copolymers, BAB block copolymers, AB block copolymers, and combinations thereof.” Even though not stated in the claim, some of these copolymers may display reverse thermal gelation properties. Having said that, the systems generated are “ . . . blends and copolymers thereof . . . ” that, as apparent to anybody skilled in the art, are inherently homogeneous solutions that will necessarily generate one homogeneous RTG solution having one Ti value and one averaged out viscosity level. Also, the only purpose of the presence of the “sparingly soluble particles is to perform as the substrate for the protein or peptide molecules to adsorb onto their surface, slowing down, therefore, their dissolution and release, In striking contrast to the invention disclosed in WO 01 76558 (D2), the unique and essential feature of the present invention is the presence of more than one polymeric reverse thermo-responsive component capable of undergoing a transition that results in a sharp increase in viscosity in response to a change in temperature at a predetermined body site, wherein said at least two components display different reverse thermal gelation behavior, The term “different reverse thermal gelation behavior” as used herein is intended to denote inter alia that the different components attain different viscosities in the human body at 37° C., that they have dfferent Ti values, meaning that their viscosities raise at different temperatures, and that one may have to dissolve over time before it starts to be RTG relevant. As also stated below, said at least two components display different reverse thermal gelation behavior, exhibiting a defined interface between them in the human body, i.e. the components have different RTG properties as a function of two parameters, namely time and position in space within the implanted sample. It is apparent, therefore that the invention disclosed in WO 01 76558 (D2) is in fundamental contrast to the invention disclosed herein.

Below, follow a few examples, to briefly illustrate the invention disclosed herein. The inventors have chosen to confine themselves to biomedical polymeric systems, even though the compositions of the present invention can be applied to other areas. Furthermore, for the sake of clarity and simplicity, and without limiting the scope of the invention in any form or fashion, the inventors have chosen to illustrate the invention hereby disclosed, by focusing on a specific biomedical application and exemplifying the invention using one particular family of RTG polymers. This, even though the multi-component systems of the present invention includes all families of RTG-displaying materials, and the compositions disclosed herein, can be applied to numerous sites in the body and can be used in fundamentally different applications. Focusing on a specific biomedical application and exemplifying the invention using one particular family of RTG polymers is intended only to illustrate preferred embodiments and should not be construed as limiting in any way or fashion, the scope of this invention, as more broadly set forth hereby.

The application selected for illustrating this invention, is their use as injectables in non-invasive or minimally invasive surgical procedures. Without limiting the scope of the invention in any form or fashion, two groups of polymeric reverse-thermoresponsive compositions have been chosen by the inventors to illustrate the present invention: (1) the first group is based on the commercially available Pluronic polyethylene oxide-polypropylene oxide-polyethylene oxide (PEO-PPO-PEO) triblocks and more specifically Pluronic F127 and (2) materials of the following generic formula [-Xₙ-A-Xₙ-E-B-E-]ₘ, where X, A, E, B, m and n are as defined above.

The term ‘viscosity’ is used to describe the fundamental characteristic of the water solutions generated by the polymeric compositions disclosed hereby, which related to the resistance of the composition to flow. For purposes of the present invention, viscosity is measured in centipoise (cP) units or Pa.s, where 1000 cP=10 Poise=1 Pa.s, as determined by a Brookfield Programmable Viscometer.

While the invention will now be described in connection with certain preferred embodiments in the following examples and with reference to the attached figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### BRIEF DESCRIPTION OF THE DRAWINGS

In the drawings:

FIG. 1 is a graphical representation of viscosity as a function of time and concentration for a composition according to the present invention; and

FIG. 2 is a graphical representation of viscosity as a function of time and

concentration for a composition according to the present invention.

### DETAILED DESCRIPTION OF PREFERRED EMBODIMENTS OF THE INVENTION

### EXAMPLE 1

A Multi-constituent RTG Composition Comprising Two Different Solutions of Pluronic F127 (PEO-PPO-PEO) with Different Concentration

The two-constituents composition described hereby, comprises one RTG polymer only, PEO-PPO-PEO triblock, Pluronic F127 (MW=12,600), the polymer being present in two different concentrations of its water solution form: 17% and 30%. The respective viscosities of their gelled solutions, at 37.2° C., were 12,200,000 cps (Ti=25.6° C.) and 71,600,000 cps (Ti=9.3° C.). The system was formed by injecting the Pluronic F127 30% water solution at a temperature below its Ti, into the gel of the 17% solution, which was kept above its Ti, typically 37° C. As it rapidly heated up, the Pluronic F127 30% solution gelled, generating a domain kind of structure within the continuous, less viscous medium formed by the 17% component. Two clearly distinct phases were initially generated, which, with time, produced a monophasic system, having the expected viscosity. By forming domains of various sizes and shapes, the rate at which the viscosity of the medium varies over time, was controlled.

### EXAMPLE 2

A Multi-constituent RTG Composition Comprising Three Different Solutions of Pluronic F127 (PEO-PPO-PEO) with Different Concentration

The three-constituents composition described hereby, comprises one RTG polymer only, PEO-PPO-PEO triblock, Pluronic F127 (MW=12,600), the polymer being present in three different concentrations of its water solution form: 20%, 25% and 30%. The respective viscosities of their gelled solutions, at 37.2° C., were 22,200,000 cps (Ti=25.6° C.), 51,000,000 cps (Ti=16.4° C.) and 71,600,000 cps (Ti=9.3° C.). The system was formed by injecting the Pluronic F127 25% water solution at a temperature below its Ti, into the gel of the 20% solution, which was kept above its Ti, typically 3° C. As it rapidly heated up, the Pluronic F127 25% solution gelled, generating a domain kind of structure within the continuous, less viscous medium formed by the 20% component. The next step consisted of injecting the Pluronic F127 30% water solution at a temperature below its Ti, into the two-component gel just generated, which was kept above the Ti of its two constituents, typically 37° C. As it rapidly heated up, the Pluronic F127 30% solution gelled, generating a second array of domains, within the continuous, less viscous medium formed by the 20% component and in addition to the domains already formed by the 25% gelled constituent. Two and three clearly distinct phases were generated throughout the process, which, with time, produced a final monophasic system, having the expected viscosity. By forming domains of various sizes and shapes, the rate at which the viscosity of the medium.

### EXAMPLE 3

A Multi-constituent RTG Composition Comprising Two Different Solutions of Polymer [-PEG6000-O-CO-O-PPG3000-]₄ with Different Concentration

A) Synthesis of Alternating [-PEG6000-O-CO-O-PPG3000-]ₙ poly(ether-carbonate)

i) Synthesis of Phosgene and Preparation of the Chloroformic Solution

The phosgene was generated by reacting 1,3,5 trioxane (15 g) with carbon tetrachloride (100 g) using aluminum trichloride (30 g) as the catalyst. The phosgene vapors were bubbled in weighed chloroform and the phosgene concentration (w/w) was calculated by weight difference (between 9% and 11%). Due to phosgene's high toxicity, the solution was handled with extreme care and all the work was conducted under a suitable hood.

Synthesis of PEG6000 Dichloroformate (CICO-O-PEG6000-O-COCI)

30.3 grams of dried PEG6000 (molecular weight 6,000) were dissolved in 50 ml dried chloroform in a 250 ml flask. 66 gram of chloroformic solution of phosgene 3% w/w (100% molar excess to PEG) were added to the PEG and the mixture was allowed to react at 60° C. for 4 h with magnetic stirring and a condenser in order to avoid solvent and phosgene evaporation. The reaction flask was connected to a NaOH trap (20% w/w solution in water/ethanol 1:1) in order to trap the phosgene that could be released during the reaction. Once the reaction was completed, the system was allowed to cool down to RT and the excess of phosgene was eliminated by vacuum. The FT-IR analysis showed the characteristic peak at 1777 cm″ belonging to the chloroformate group vibration.

iii) Synthesis of Alternating 1-PEG6000-O-CO-O-PPG3000-1 poly(ether-carbonate)

15.2 grams of dried PPG3000 (molecular weight 3,000) were added to CICO-PEG6000-COCI produced in a) at RT. The mixture was cooled to 5° C. in an ice bath and 6.3 grams pyridine dissolved in 20 ml chloroform were added dropwise over a 15 min period. Then, the temperature was allowed to heat up to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained. The material displayed a melting endotherm at 53.5° C. and the FT-IR analysis showed the characteristic carbonate group peak at 1746 cm⁻¹. The molecular weight of the polymer produced was Mₙ 36,400 (Mw/Mn=1.28), as determined by GPC. The PEG/PPG block ratio in the final product was determined by ¹H-NMR using a calibration curve obtained from different blends having various PEG6000/PPG3000 ratios and was 1.78, whereas the PEO/PPO ratio was 4.7.

A) Preparation of The Two Multi-component Polymeric System

The two-constituents composition described hereby, comprises one RTG polymer only, [-PEG6000-O-CO-O-PPG3000-]₄, the polymer being present in two different concentrations of its water solution form: 10% and 20%. The respective viscosities of their gelled solutions, at 37.2 degrees centigrades, were 1,600,000 cps and 58,600,000 cps. The system was formed as described above, in Example 1. Two clearly distinct phases were initially generated, which, with time, produced a monophasic system, having the expected viscosity. By forming domains of various sizes and shapes, the rate at which the viscosity of the medium varies over time, was controlled.

### EXAMPLE 4

A Multi-constituent RTG Composition Comprising Three Different Solutions of Polymer [-PEG6000-O-CO-O-PPG3000-]₄ with Different Concentration

A) Synthesis of Alternating [-PEG6000-O-CO-O-PPG3000-]ₙ poly(ether-carbonate

The synthesis of the polymer [-PEG6000-O-CO-O-PPG3000-]₄ was described in Example 3A).

Preparation of The Three Multi-component Polymeric System

The three-constituents composition described hereby, comprises one RTG polymer only, [-PEG6000-O-CO-O-PPG3000-]₄, the polymer being present in three different concentrations of its water solution form: 10%, 15% and 20%. The respective viscosities of their solutions, at 37.2 degrees centigrades, were 1,600,000 cps, 13,200,000 cps and 58,600,000. The system was formed as described above, in Example 2. Three clearly distinct phases were initially generated, which, with time, produced a monophasic system, having the expected viscosity. By forming domains of various sizes and shapes, the rate at which the viscosity of the medium varies over time, was controlled.

### EXAMPLE 5

A Multi-constituent RTG Composition Comprising Two Solutions of Polymer [-PEG4000-O-CO-O-PPG4000-]₅ with Two Different Concentrations

A) Synthesis of Alternating [-PEG4000-O-CO-O-PPG4000-]ₙ poly(ether-carbonate) i) Synthesis of Phosgene and Preparation of The Chloroformic Solution

The synthesis of phosgene and preparation of the chloroformic solution were described in Example 4A)i).

A) Synthesis of PEG4000 Dichloroformate (CICO-O-PEG4000-O-COCI)

The procedure described in example 4A)ii) was essentially repeated, except that 20.2 grams (0.005 mol) PEG4000 (molecular weight 4,000) and 20 grams of the chloroformic solution of phosgene 7.7% w/w (100% molar excess to PEG), were used. The FT-IR analysis showed the characteristic peak at 1777 cm⁻¹ belonging to the chioroformate group vibration.

iii) Synthesis of Alternating [-PEG4000-O-CO-O-PPG4000-]ₙ poly(ether-carbonate)

The procedure in example 4A)iii) was essentially repeated, except that 20.1 grams (0.005 mol) PEG4000 (molecular weight 4,000) and 7.9 grams pyridine were used. A light yellow powder was obtained. The product showed T_{g} at −74° C. and Tₘ at 50° C. and FT-IR analysis showed the characteristic carbonate peak at 1746 cm⁻¹. The

molecular weight of the polymer produced was Mₙ 25,500 (M_{w}/Mₙ=1.53), as determined by GPC. The PEG/PPG block ratio, as determined by ¹H-NMR, was 1.27, whereas the molar ratio PEO/PPO was 1.67.

Preparation of The Two Multi-component Polymeric System

The two-constituents composition described hereby, comprises one RTG polymer only, [-PEG4000-O-CO-O-PPG4000-]₄, the polymer being present in two different concentrations of its water solution form: 5% and 15%. The respective viscosities of their gelled solutions, at 37.2° C., were 512.000 cps and 37.500.000 cps. The system was formed as described above, in Example 1. Two clearly distinct phases were initially generated, which, with time, produced a monophasic system, having the expected viscosity. By forming domains of various sizes and shapes, the rate at which the viscosity of the medium varies over time, was controlled.

### EXAMPLE 6

A Multi-constituent RTG Composition Comprising Three Solutions of Polymer

[-PEG4000-O-CO-O-PPG4000-]₄ with Three Different Concentrations

A Synthesis of Alternating [-PEG4000-O-CO-O-PPG4000-]ₙ poly(ether-carbonate)

The synthesis of the polymer [-PEG4000-O-CO-O-PPG4000-]₄ was described in Example 5A).

Preparation of The Three Multi-component Polymeric System

The three-constituents composition described hereby, comprises one RTG polymer only, [-PEG4000-O-CO-O-PPG4000-]₄, the polymer being present in three different concentrations of its water solution form: 5%, 10% and 15%. The respective viscosities of their gelled solutions, at 37.2° C., were 512,000 cps, 10,800,000 cps and 37,500,000 cps. The system was formed as described above, in Example 2. Three clearly distinct phases were initially generated, which, with time, produced a monophasic system, having the expected viscosity. By forming domains of various sizes and shapes, the rate at which the viscosity of the medium varies over time, was controlled.

### EXAMPLE 7

A Multi-constituent Composition Comprising Two RTG Polymers of the Following Formulae: [-PEG4000-O-CO-O-PPG4000-]₄ and [-PEG6000-O-CO-O-PPG4000-]₄

A Synthesis of Alternating [-PEG6000-O-CO-O-PPG4000-]ₙ poly(ether-carbonate) i) Synthesis of Phosgene and Preparation of the Chloroformic Solution

The synthesis of phosgene and preparation of the chloroformic solution were described in Example 3A)i).

A) Synthesis of PEG6000 Dichloroformate (CICO-O-PEG6000-O-COCI)

The synthesis of PEG6000 dichloroformate was described in Example 3A)ii).

iii) Synthesis of Alternating [-PEG6000-O-CO-O-PEG4000-]ₙ poly(ether-carbonate)

The procedure in example 3A)iii) was essentially repeated, except that 20.3 grams (0.0051 mol) PPG4000 (molecular weight 4,000) and 7.9 grams pyridine were used. The product was a light yellow powder, which showed a T_{g} at −73° C. and Tₘ at 45° C., and FT-IR analysis showed the carbonate characteristic peak at 1746 cm⁻¹. The molecular weight of the polymer produced was Mₙ 29,200 (M_{w}/Mₙ=1.35), as determined by GPC. The PEG/PPG block ratio determined by ¹H-NMR using a calibration curve obtained from different ratio PEG3400/PPG4000 blends and was. The polymer produced presented Mₙ 12,500 (M_{w}/Mₙ=2.38). The PEG/PPG block molar ratio determined by ¹H-NMR using a calibration curve obtained from different ratio PEG4000/PPG4000 blends and was 1.15, whereas the molar ratio PEO/PPO was 1.3.

B) Synthesis of Alternating [-PEG4000-O-CO-O-PPG4000-]ₙ poly(ether-carbonate)

The syntheis of the polymer [-PEG4000-O-CO-O-PPG4000-]₄ was described in Example 5A).

Preparation of the Two Multi-component Polymeric System

The two-constituents composition described hereby, comprises two RTG polymers, [-PEG4000-O-CO-O-PPG4000-]₄ and [-PEG6000-O-CO-O-PPG4000-]₄. The concentration of the polymers was 5% and 10%, respectively, and the viscosity levels attained by their gelled solutions at 37.2° C., were 512,000 cP and 43,800,000 cP, respectively. The system was formed as described above, in Example 1. Two clearly distinct phases were initially generated, which, with time, produced a monophasic system, having the expected viscosity. By forming domains of various sizes and shapes, the rate at which the viscosity of the medium varies over time, was controlled.

### EXAMPLE 8

A Multi-constituent Composition Comprising Two RTG Polymers of the Following Formulae: [-PEG6000-O-CO-O-PPG3000-]₄ and [-PEG4000-O-CO-O-PPG4000-]₄

A) Synthesis of Alternating ]-PEG6000-O-CO-O-PPG3000-]ₙ poly(ether-carbonate)

The synthesis of the polymer [-PEG6000-O-CO-O-PPG3000-]₄ was described in Example 3A).

Synthesis of alternating [-PEG4000-O-CO-O-PPG4000-]ₙ poly(ether-carbonate)

The synthesis of the polymer [-PEG4000-O-CO-O-PPG4000-]₄ was described in Example 5A).

Preparation of the Two Multi-component Polymeric System

The two-constituents composition described hereby, comprises two RTG polymers, [-PEG6000-O-CO-O-PPG3000-]₄ and [-PEG4000-O-CO-O-PPG4000-]₄. The concentration of the polymers was the same, 10%, and the viscosity levels attained by their gelled solutions at 37.2° C., were 1,600,000 cP and 10,800,000 cP, respectively. The system was formed as described above, in Example 1. Two clearly distinct phases were initially generated, which, with time, produced a monophasic system, having the expected viscosity. By forming domains of various sizes and shapes, the rate at which the viscosity of the medium varies over time, was controlled.

### EXAMPLE 9

A Multi-constituent Composition Comprising Two RTG Polymers of The Following Formulae: Pluronic F127 (PEO-PPO-PEO) and [-PEG4000-O-CO-O-PPG4000-]₄

A) Synthesis of Alternating [-PEG4000-O-CO-O-PPG4000-]ₙ poly(ether-carbonate)

The synthesis of the polymer [-PEG4000-O-CO-O-PPG4000-]₄ was described in Example 5A).

Preparation of the Two Multi-component Polymeric System

The two-constituents composition described hereby, comprises two RTG polymers, Pluronic F127 (PEO-PPO-PEO) and [-PEG4000-O-CO-O-PPG4000-]₄. The concentration of the polymers were 30% and 5%, respectively, and the viscosity levels attained by their gelled solutions at 37.2° C., were 71,600,000 cP and 512,000 cP, respectively. The system was formed as described above, in Example 1. Two clearly distinct phases were initially generated, which, with time, produced a monophasic

system, having the expected viscosity. By forming domains of various sizes and shapes, the rate at which the viscosity of the medium varies over time, was controlled.

### EXAMPLE 10

A Multi-constituent RTG Composition Comprising Polymers of the Following General Formula: Random [-PEG6000-O-CO-O-PPG3000-]₄

A) Synthesis of Alternating [-PEG6000-O-CO-O-PPG3000-]ₙ poly(ether-carbonate)

The synthesis of the polymer [-PEG6000-O-CO-O-PPG3000-]₄ was described in Example 3A).

B) Preparation of the Two Multi-component Polymeric System

The two-constituents composition described hereby, comprises one RTG polymer only, the random [-PEG6000-O-CO-O-PPG3000-]₄ polymer being present in two different forms: liquid and solid. The gelled solution 4% w/w, at 37.2° C., has an initial viscosity of 512,000 cP. Then polymer in solid form was added in order to achieve a final 10% w/w solution, when dissolved. After that the system was incubated at 30° C. during 15 hours. The viscosity achieved was at 37° C. 30,000,00 cP.

### EXAMPLE 11

A Multi-constituent RTG Composition Comprising Pluronic F127 in Solution and Solid Form

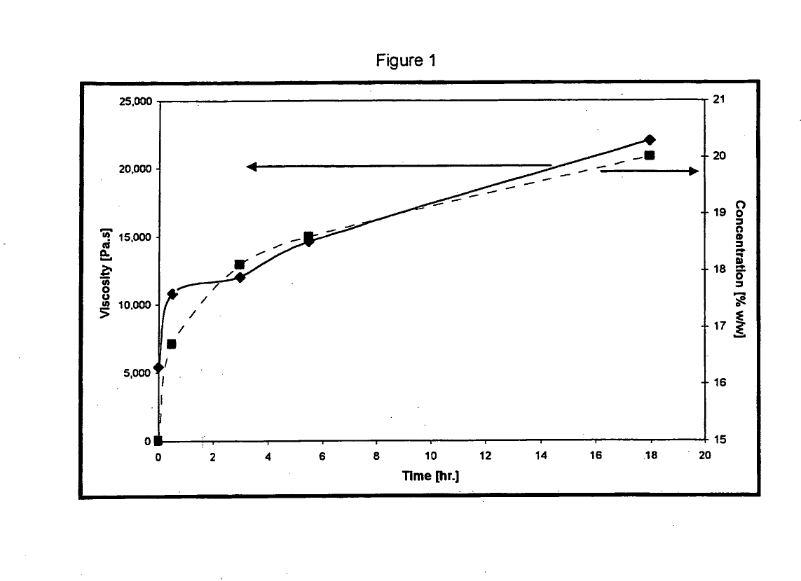

The two-constituents composition described hereby, comprises one RTG polymer only, the Pluronic F127 polymer being present in two different forms: liquid and solid. The gelled 15% w/w solution, at 37.2° C., has an initial viscosity of 5,400 Pa.s. Then polymer in solid form was added in order to achieve a final 20% w/w solution, when dissolved. After that the system was incubated at 37° C. during different periods of time. The viscosity achieved by the liquid phase and the corresponding concentration is described in the graph of FIG. 1.

### EXAMPLE 12

A Multi-constituent RTG Composition Comprising Pluronic F127 in Solution and Solid Form

The two-constituents composition described hereby, comprises one RTG polymer only, the Pluronic F127 polymer being present in two different forms: liquid and solid. The geled solution 15% w/w, at 37.2° C., has an initial viscosity of 5,400 Pa.s.

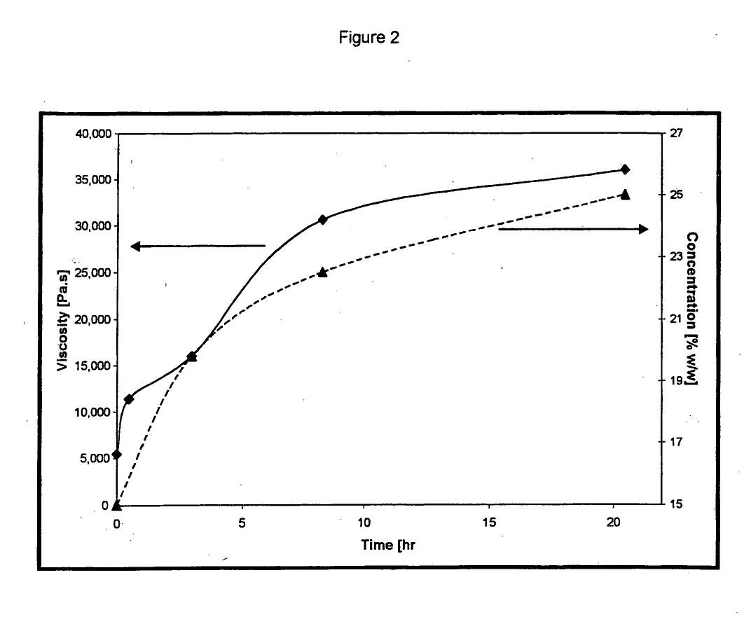

Then polymer in solid form was added in order to achieve a final 25% w/w solution, when dissolved. After that the system was incubated at 37° C. during different periods of time. The viscosity achieved by the liquid phase and the corresponding concentration is described in the graph of FIG. 2:

It will be evident to those skilled in the art that the invention is not limited to the details of the foregoing illustrative examples and that the present invention may

1) A multi-component environmentally responsive polymeric system, comprising at least two environmentally responsive polymeric components capable of undergoing a transition that results in a sharp increase in viscosity in response to a change in temperature at a predetermined body site, wherein said at least two components display different reverse thermal gelation behavior in the human body.

2) The multi-component responsive polymeric system of claim 1, wherein said increase in viscosity takes place when the system is heated up from a lower temperature to body temperature.

3) The multi-component responsive polymeric system of claim 1, wherein said responsive polymeric system displays an initial sharp increase in viscosity at insertion time, followed by additional changes in viscosity in situ, as a function of time.

4) The multi-component responsive polymeric system of claim 1, wherein said responsive polymeric system comprises at least one biodegradable responsive component.

5) The multi-component responsive polymeric system of claim 1, wherein each of said components is comprised of the same polymer and said components are present in different concentrations or states and as a result of said different concentrations or states display different reverse thermal gelation behavior.

6) The multi-component responsive polymeric system of claim 1, adapted for insertion into the human body, wherein at least one of said components in a water solution form at the time of insertion.

7) The multi-component responsive polymeric system of claim 1, adapted for insertion into the human body wherein each responsive polymeric component is in a water solution form at the time of insertion.

8) The multi-component responsive polymeric system of claim 1, adapted for insertion into the human body, wherein at least one of said components is in a solid form at the time of insertion.

9) The multi-component responsive polymeric system of claim 6, wherein said at least one component present at the time of insertion in a water solution form is adapted to generate a continuous gel phase or independent or interconnected domains of vrious sizes, shapes and spatial orientations within the system at a predetermined body site, all of these characteristics being able to change in situ over time affecting, therefore, the properties of the whole system.

10) The multi-component responsive polymeric system of claim 8, wherein said at least one solid responsive polymeric component is a solid appearing in a diversity of shapes, sizes and geometries selected from a group consisting of spheres, particles, capsules, fibers, ribbons, films, meshes, fabrics, non-woven structures, foams, honey-comb structures, porous structures, and combinations thereof, each of them having the possibility of being solid, porous, hollow, and/or combinations thereof wherein said at least one solid component comprises at least one reverse thermo-responsive polymer and wherein said solid components are engineered so that a diversity of spatial arrays are obtained, dispersed homogeneously or heterogeneously, isotropically or anisotropically within the system, generating macro, micro or nanoscopic independent or interconnected domains within the system

11) The multi-component responsive polymeric system of claim 1, wherein each of said responsive components is selected from a group consisting poly(ethylene oxide)-poly(propylene oxide)-poly(ethylene oxide) (PEO-PPO-PEO) triblocks, random or alternating reverse thermo-responsive PEO-PPO block copolymers, N-alkyl substituted acrylamides, cellulose derivatives and combinations thereof.

12) The multi-component responsive polymeric system of claim 1, wherein said responsive component is selected from a group consisting of a polyoxyalkylene polymer, a block copolymer comprising polyethylene oxide (PEO) and polypropylene oxide (PPO) selected from a group consisting of a diblock, a triblock or a multiblock, a segmented block copolymer comprising polyethylene oxide (PEO) and polypropylene oxide (PPO) chains, wherein said PEO and PPO chains are connected via a chain extender, a poly(alkyl-co-oxyalkylene) copolymer having the formula R—(OCH₂CH)n-OH, where R is an hydrophobic monofunctional segment selected from a group consisting of poly(tetramethylene glycol), poly(caprolactone), poly(lactic acid), poly(siloxane) and combinations thereof, a poly(alkyl-co-oxyalkylene) copolymer having the formula [—R′—(OCH2CH)n-O]pH, where R′ is a bifunctional or multifunctional hydrophobic segment, a poly(N-alkyl substituted acrylamide)s, cellulose and cellulose derivatives and combinations thereof.

13) The multi-component responsive polymeric system of claim 1, wherein said responsive polymeric system comprises at least two different environmentally responsive polymeric components.

14) The multi-component responsive polymeric system of claim 1, wherein said responsive polymeric system further comprises other polymers that are responsive to other stimuli selected from a group consisting of pH, ionic strength, electric and magnetic fields, ultrasound radiation, fluids and biological species and combinations thereof.

15) The multi-component responsive polymeric system of claim 1, wherein said responsive polymeric system comprising at least two environmentally responsive polymeric components comprises other non-responsive materials, organic, inorganic or biological, polymeric or not, that fulfill other chemical, physical, rheological, mechanical or biological roles.

16) The multi-component responsive polymeric system of claim 1, wherein at least one of said responsive polymeric components is crosslinked.

17) The responsive polymeric system of claim 16, wherein said crosslinked component is crosslinked in the body.

18) The responsive polymeric system of claim 16, wherein said crosslinking is temporary so that the system is able to essentially revert in the body, to its non-crosslinked state.

19) The responsive polymeric system of claim 1, wherein at least one of said responsive components contains a molecule or molecules, displaying biological activity, to be delivered into the body following a unimodal or multimodal release kinetics.

20) The responsive polymeric system of claim 1, wherein at least one of said responsive components contains organic or inorganic materials of biological source.

21) The responsive polymeric system of claim 1, wherein at least one of said responsive components contains living cells of at least one type.

22) The responsive polymeric system of claim 1, wherein at least one of said responsive componenst contains components of biological origin selected from a group consisting of elastin, a collagenous material, albumin, a fibrinous material, demineralized tissue or an acellular tissue matrix and combinations thereof.

23) The multi-component responsive polymeric system of claim 1, whenever used as matrices for the unimodal or multimodal controlled release of biologically active agents, as sealants, as coatings and lubricants, as transient barriers for the prevention of post-surgical adhesions, in the area of Tissue Engineering and the field of Gene Therapy.

24) The multi-component responsive polymeric system of claim 1, whenever used as both the matrix and the scaffold in the area of ex vivo as well as in vivo Tissue Engineering comprising one or more types of cells.

26) The multi-component responsive polymeric system of claim 1, wherein said at least two components display different reverse thermal gelation behavior, displaying initially a defined Interface therebetween.

26) The multi-component responsive polymeric system of claim 6, wherein at least one of said components that is in a water solution form at the time of insertion, polymerizes and/or crosslinks after insertion into the human body.

27) The multi-component responsive polymeric system of claim 15, wherein said non-responsive material polymerizes and/or crosslinks after insertion into the human body.
